(19) United States
(12) Patent Application Publication
(10) Pub. No.: US 2026/0335478 A1
(43) Pub. Date: Oct. 29, 2026

(54) APPARATUS AND METHOD FOR IRRADIATING A BIOLOGICAL FLUID

(71) Applicant: Maco Pharma, Mouvaux (FR)

(72) Inventors: Pierre-Eloi BONTINCK, Mouvaux (FR); Cecile BIRCK, Mouvaux (FR); Sebastien VANHELST, Mouvaux (FR)

(21) Appl. No.: 18/997,193

(22) PCT Filed: Jul. 18, 2023
(86) PCT No.: PCT/EP2023/069968

§ 371 Date: Jan. 21, 2025

(30) Foreign Application Priority Data
Jul. 21, 2022 (FR) FR2207464

(51) Int. Cl. A61L 2/104; A61L 103/09; A61M 1/36

(52) U.S. Cl. CPC A61L 2/104; A61M 1/3681; A61L 2103/09; A61L 2202/11; A61L 2202/122; A61L 2202/14

(57) ABSTRACT

The invention relates to an apparatus (21) for the treatment, by electromagnetic irradiation at a predetermined dose, of a biological fluid contained in an irradiation receptacle, the irradiation receptacle having at least one wall which is permeable to the electromagnetic radiation. The apparatus comprises: a recess (22) which is configured to receive the irradiation receptacle (22) containing the biological fluid; one or more light sources which emit the electromagnetic radiation; an information collection device (34) which is configured to collect information regarding a parameter of the irradiation receptacle; and a control unit which is configured to: (i) obtain information regarding a parameter of the irradiation receptacle; (ii) determine a target irradiation dose on the basis of the information regarding the parameter and the predetermined dose; and (iii) irradiate the irradiation receptacle with the target irradiation dose, the information regarding the parameter of the irradiation receptacle comprising the transmittance value of the wall of the irradiation receptacle.

The present invention concerns an apparatus and a method for the treatment by electromagnetic irradiation of a biological fluid, as well as an irradiation receptacle and a system comprising said apparatus and said irradiation receptacle.

It applies to the medical and biomedical field, in particular to the field of blood transfusion, and even more specifically to the field of blood processing.

In transfusion, blood is first collected from a donor and then separated by centrifugation into different blood products such as red blood cells concentrates, plasma and platelet concentrates. To improve the quality and safety of these different blood products, they may undergo various treatments, such as leukocyte removal and/or pathogen reduction.

Document WO 2007/076834 describes a method for reducing pathogens such as bacteria, viruses and/or leukocytes from platelet concentrates. In this method, a bag containing a platelet concentrate is irradiated with UV radiation at a predetermined dose and under agitation. This method has the advantage, over other commercial pathogen inactivation methods, such as Intercept method (Cerus) and Mirasol method (TerumoBCT), of not using a photosensitive agent such as amotosalen or riboflavin.

In these light-based methods, and particularly in the absence of a photosensitive agent, it is important to ensure that the blood product to be treated receives the necessary and sufficient dose of light to reduce the rate of pathogens to an acceptable level, without damaging the biological properties of the blood product.

Document WO 2009/000445A1 describes an irradiation apparatus for treating a biological fluid with UV-C radiation according to the method described in document WO 2007/076834. The irradiation apparatus comprises a housing, a stirring plate for supporting the irradiation bag and a light source arranged on either side of said plate for irradiating the bag. In order to control the irradiation dose applied to the biological fluid, optical sensors are provided in the vicinity of the light source.

WO 2019/133929A1 describes an irradiation apparatus for specifically implementing the Intercept method for reducing pathogens in a biological fluid using UVA light and amotosalen. According to one embodiment, the apparatus comprises a control circuit configured to establish a treatment profile comprising the duration and intensity of irradiation as a function of the characteristics of the biological fluid to be treated. These characteristics include the volume, type or temperature of the biological fluid to be irradiated. They are supplied by the operator or determined by sensors arranged in the irradiation apparatus, or obtained by a barcode reader.

In these two prior art documents, the optical properties of the receptacle containing the biological fluid to be irradiated are not taken into account when determining the irradiation dose to be delivered by the irradiation apparatus.

Document WO 2008/034476 describes a bag system suitable for implementing the method for reducing pathogens by UV irradiation described in document WO 2007/076834. The bag system essentially comprises an irradiation bag made of ethylene-vinyl acetate copolymer (EVA) connected to a platelet concentrate storage bag made of plasticized polyvinyl chloride (PVC).

In this document, it is stated that the absorption of UV light by EVA is influenced by the degree of polymerization and crosslinking of the EVA.

Other intrinsic factors influence the UV transmittance of an EVA sheet, such as the relative proportion of ethylene and vinyl acetate units, the thickness of the film or the surface finish thereof. Extrinsic factors affecting UV transmittance include heat, humidity, the presence of oxygen and exposure to UV rays.

Thus, after numerous tests, the applicant has found that the UV transmittance of an EVA film used to manufacture irradiation bags can vary by up to 10% between different film production batches. The final irradiation bags therefore do not all have the same UV transmittance.

As a result, there is a risk that the biological fluid to be treated may not receive a sufficient dose of light to inactivate pathogens, or may receive a too high dose, which may adversely affect the quality of the biological fluid irradiated.

The invention allows to overcome these drawbacks.

According to a first aspect, the invention proposes a method for the treatment by electromagnetic irradiation at a predetermined dose of a biological fluid contained in an irradiation receptacle, said irradiation receptacle having at least one wall permeable to said electromagnetic radiation, the method comprising the following steps:
(i) obtaining information regarding a parameter of said irradiation receptacle,
(ii) determining a target irradiation dose on the basis of the information regarding said parameter and said predetermined dose, and
(iii) irradiating the irradiation receptacle with said target irradiation dose, said information regarding the parameter of the irradiation receptacle comprising the value of the transmittance of the wall of the irradiation receptacle.

According to a second aspect, the invention relates to an apparatus for the treatment by electromagnetic irradiation at a predetermined dose of a biological fluid contained in an irradiation receptacle, said irradiation receptacle having at least one wall permeable to said electromagnetic radiation, said apparatus comprising a recess configured to receive said irradiation receptacle containing the biological fluid, one or more light sources emitting said electromagnetic radiation, an information collection device configured to collect information regarding a parameter of said irradiation receptacle, and a control unit configured to (i) obtain the information regarding a parameter of said irradiation receptacle, (ii) determine a target irradiation dose on the basis of said parameter and said predetermined dose, and (iii) irradiate the irradiation receptacle with said target irradiation dose, the information regarding the parameter of the irradiation receptacle comprising the transmittance value of the wall of the irradiation receptacle.

A third aspect of the invention relates to an irradiation receptacle having at least one wall permeable to electromagnetic radiation and comprising information regarding a parameter of said irradiation receptacle, said information comprising the transmittance value of the wall of the irradiation receptacle.

According to a fourth aspect, the invention comprises a system comprising an apparatus according to the second aspect of the invention and an irradiation receptacle according to the third aspect of the invention.

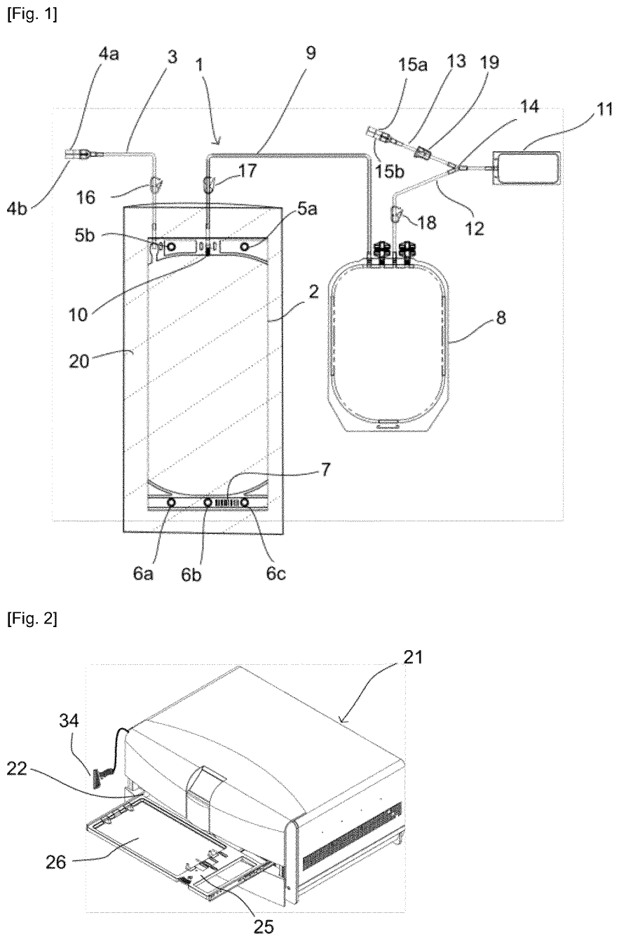

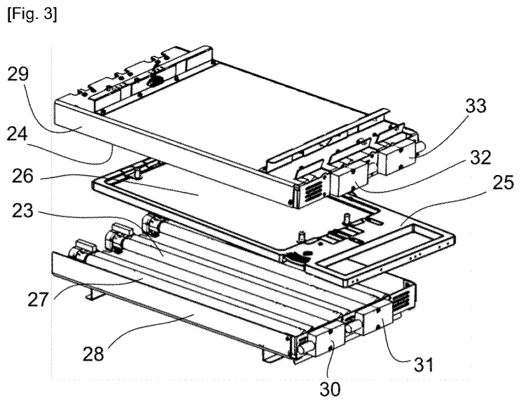

The annexed drawings illustrate the invention:

[FIG. 1] schematically shows a bag system comprising an irradiation receptacle according to one embodiment of the invention.

[FIG. 2] schematically shows an irradiation apparatus according to one embodiment of the invention.

[FIG. 3] shows a partial view of the irradiation apparatus shown in [FIG. 2].

According to one aspect, the invention relates to a method for the treatment by electromagnetic irradiation at a predetermined dose of a biological fluid contained in an irradiation receptacle, said irradiation receptacle having at least one wall permeable to said electromagnetic radiation. The method comprises the following steps:
(i) obtaining information regarding a parameter of said irradiation receptacle, said information comprising the transmittance value of the wall of the irradiation receptacle,
(ii) determining a target irradiation dose on the basis of said information regarding said parameter and said predetermined dose, and
(iii) irradiating the irradiation receptacle with said target irradiation dose.

The invention ensures that the biological fluid contained in the irradiation receptacle receives the appropriate dose of electromagnetic irradiation for effective treatment, taking into account the optical properties of the irradiation receptacle.

In one embodiment, the biological fluid is whole blood or a blood product obtained by filtration and/or centrifugation of whole blood, such as plasma, platelet-rich plasma, red cell concentrate or platelet concentrate. For example, the biological fluid is a platelet concentrate obtained by apheresis or derived from a mixture of buffy coats.

In order to reduce pathogens such as viruses, bacteria, parasites and/or leukocytes in blood or a blood product, it is known to apply electromagnetic radiation to the blood or blood product, in the presence or absence of a photosensitive agent.

By electromagnetic radiation, we mean non-ionizing radiation with wavelengths ranging from 10 nm to 10 μm, i.e. visible, ultraviolet (UV) and/or infrared radiation.

According to one embodiment, the electromagnetic radiation is UV radiation with wavelengths ranging from 200 to 340 nm. More particularly, the UV radiation is UV-C radiation with wavelengths ranging from 200 to 280 nm. Even more particularly, the UV-C radiation has a wavelength of the order of 254 nm.

In one embodiment, the electromagnetic radiation dose ranges from 0.01 J/cm² to 10 J/cm².

For example, for UV-C radiation, the UV-C radiation dose is a dose ranging from 0.01 J/cm² to 2 J/cm², particularly ranging from 0.05 J/cm² to 0.5 J/cm², and even more particularly is of the order of 0.2 J/cm². In particular, the biological fluid to be irradiated does not comprise a photosensitive agent.

To enable it to be treated, the biological fluid is contained in an irradiation receptacle having at least one wall that is permeable to said electromagnetic radiation. In one embodiment, the irradiation receptacle is an irradiation bag comprising at least one wall of a flexible film made of a material permeable to electromagnetic radiation.

[FIG. 1] shows an example of a bag system 1 comprising such an irradiation receptacle 2, in the form of an irradiation bag, for the treatment by electromagnetic irradiation of a biological fluid.

The bag system 1 for irradiation by electromagnetic radiation of a biological fluid comprises at least one irradiation bag 2 designed to contain the biological fluid to be irradiated. The irradiation bag 2 has at least one wall permeable to electromagnetic radiation. In particular, the irradiation bag is made of a material permeable to said electromagnetic radiation.

In a very specific embodiment, the bag system 1 allows the irradiation of a platelet concentrate contained in the irradiation bag 2 by UV-C radiation.

For example, the irradiation bag 2 is made of EVA. Alternatively, the irradiation bag 2 is made of a material permeable to UV radiation, such as polymethylpentene, polychlorotrifluoroethylene, perfluoroalkoxy, fluorinated ethylenepropylene, ethylene tetrafluoroethylene, ethylene chlorotrifluoroethylene or polyvinylidene fluoride. The material of the irradiation bag 2 is phthalate-free.

In particular, the irradiation bag 2 is formed from a sheath or by assembling two sheets permeable to UV radiation. The sheath or each of the sheets forming the wall(s) of the irradiation receptacle has a UV-C transmittance greater than 60%, particularly greater than 65%. Even more particularly, the UV-C transmittance of the sheath or of each of the sheets is less than 90%, particularly less than 85%.

Transmittance is the percentage ratio between the intensity of transmitted electromagnetic radiation and the incident intensity. The UV transmittance of a wall of the receptacle is the percentage ratio between the intensity of UV radiation transmitted through the wall of the receptacle and the incident intensity.

Transmittance is determined spectrophotometrically. Advantageously, an integrating sphere spectrophotometer is used to take account of material diffusion.

In a particular embodiment, the irradiation bag 2 is free of photosensitive agents. In other words, the irradiation bag 2 does not contain a photosensitive agent such as riboflavin, a psoralen derivative or a phenothiazine derivative.

According to [FIG. 1], the irradiation bag 2 is in fluid communication with a first tube 3. The first tube 3 is fitted at its end with two vents 4a, 4b, each comprising an air-permeable filter membrane forming a sterile barrier. These vents 4a, 4b enable the bag system 1 to be sterilized by gas.

The irradiation bag 2 further comprises openings 5a,5b,6a;6b,6c made in one or more peripheral edges of the irradiation bag 2 for suspending and/or holding said irradiation bag 2.

The bag system 1 also includes a storage bag 8 for collecting and storing the treated blood component. This storage bag 8 is in fluid communication with the irradiation bag 2 via a second tube 9.

A removable plug 10 is arranged in an access opening of the irradiation bag 2, so as to close off fluid communication between the inner volume of the irradiation bag 2 and the second tube 9 leading to the storage bag 8. The removable plug 10 can slide into the inner volume of the irradiation bag 2 to allow fluid communication between the irradiation bag 2 and the storage bag 8.

The bag system 1 also includes a sampling bag 11 in fluid communication with the storage bag 8 via a third tube 12.

A fourth tube 13 is connected at one end to the third tube 12 via a three-way connector 14. The other end of this fourth tube 13 is fitted with two vents 15a, 15b, each comprising an air-permeable filter membrane forming a sterile barrier. These vents 15a, 15b enable the bag system 1 to be sterilized by gas.

Each of the tubes 3,9,12,13 is fitted with a clamp 16,17,18,19 to selectively control the opening and closing of the liquid flow in each of these tubes 3,9,12,13.

The bag system 1 advantageously comprises at least one protective sleeve 20 covering said irradiation bag 2. The protective sleeve 20 is made of a phthalate-free formulated polymer material. The irradiation bag 2 is arranged in the protective sleeve 20 so as to envelop it.

The function of the protective sleeve 20 is to protect the irradiation bag 2 from external substances that may adversely affect the transmittance, in particular the UV transmittance, and even more particularly the UV-C transmittance, of said irradiation bag. The irradiation bag 2 is removed from this protective sleeve 20 when it is treated by electromagnetic radiation, before it is inserted into the irradiation apparatus.

According to the invention, the irradiation receptacle 2 comprises information regarding a parameter of said irradiation receptacle, said information comprising the transmittance value of at least one wall of the irradiation receptacle 2.

In particular, the information includes the value of the UV or UV-C transmittance of a wall of the irradiation receptacle 2.

This transmittance value of the irradiation receptacle is variable information specific to each irradiation receptacle, i.e. it assumes or can assume a distinct value depending on the irradiation receptacle used to treat the biological fluid.

Indeed, UV or UV-C transmittance varies according to the manufacturing batch of the sheath or sheets used to make the irradiation receptacle.

In a particular example, the transmittance value of a wall of the irradiation receptacle is the UV or UV-C transmittance value of the sheath or sheets forming the irradiation receptacle.

This transmittance value is the transmittance value of the sheath or sheets forming the irradiation receptacle, determined before the bag is manufactured, i.e. on the raw material used to manufacture the receptacle.

Alternatively, the transmittance value is the transmittance value of the sheath or sheets forming the irradiation receptacle, determined on the finished product, after manufacture of the irradiation receptacle.

In one embodiment, the information regarding the parameter of the irradiation receptacle is included in an information storage and retrieval means, such as a human-readable inscription, barcode, QR code or RFID chip.

An advantageous embodiment for securing information is to transcribe it into a code or symbol system comprising a set of numbers and/or letters.

The information storage and retrieval means is associated with the irradiation receptacle or with another element of the bag system comprising the irradiation receptacle. For example, the inscription or barcode or QR code comprising the information is printed directly on a wall of the irradiation receptacle. In another example, the RFID chip is integrated into the irradiation receptacle 2.

Alternatively, the information storage and retrieval means is included in a tag or label associated with the irradiation receptacle or another element of the bag system comprising the irradiation receptacle.

For example, the inscription, barcode or QR code is printed on a label stuck to the irradiation receptacle.

According to [FIG. 1], the irradiation bag 2 comprises a barcode 7 that is printed and arranged on a peripheral edge of the irradiation bag 2, in particular between two openings 6b, 6c for suspending the irradiation bag 2. The barcode 7 includes the UV or UV-C transmittance value of the irradiation bag 2.

In relation with the drawings and according to one aspect of the invention, an apparatus is now described for the treatment, by electromagnetic irradiation at a predetermined dose, of a biological fluid contained in an irradiation receptacle

This apparatus is particularly suitable for carrying out the method for the treatment by electromagnetic irradiation at a predetermined dose of a biological fluid contained in an irradiation receptacle disclosed above.

With reference to [FIG. 2] and [FIG. 3], the apparatus 21 comprises a recess 22 configured to receive an irradiation bag 2 containing the biological fluid to be irradiated, one or more light sources 23,24 emitting electromagnetic radiation and a control unit (not shown).

In one embodiment, the recess 22 comprises a plate 25 on which the irradiation receptacle 2 is intended to be placed. Advantageously, the central part 26 of the plate 25 is permeable to electromagnetic radiation, so as to enable irradiation of the irradiation receptacle 2 from above and below.

In particular, the central part 26 of the plate 25 is permeable to UV radiation and even more particularly to UV-C radiation. For example, the central part 26 of the plate 25 is made of quartz.

Advantageously, the plate 25 is a stirring plate. The stirring plate 25 performs an orbital rotation movement, for example, so as to evenly irradiate the contents of the irradiation receptacle 2.

A motor (not shown) drives the rotation of the plate 25.

The apparatus 21 also includes one or more light sources 23, 24 emitting electromagnetic radiation, in particular UV radiation, and even more particularly UV-C radiation.

In one embodiment, the apparatus 21 comprises two light sources 23, 24 on either side of the recess 22, in particular on either side of the plate 25. Each of the light sources 23, 24 comprises a plurality of lamps 27 emitting electromagnetic radiation, in particular UV radiation, and even more particularly UV-C radiation.

For example, the apparatus 21 comprises two light sources 23,24 with three lamps 27 each, arranged on either side of the plate 25 to irradiate the irradiation receptacle 2 from above and below.

Alternatively, the light source(s) is/are composed of one or more light-emitting diode panels.

In one embodiment, reflectors 28,29 are provided to reflect the light emitted by the light source(s) 23,24 towards the irradiation receptacle 2.

In order to control the irradiation dose delivered to the irradiation receptacle 2, the apparatus comprises one or more optical sensors 30,31,32,33 arranged at the level of the light source(s) 23,24. These optical sensors 30,31,32,33 detect the intensity of the irradiation emitted by the light source(s) 23,24.

The apparatus also includes a control unit. According to one embodiment, the control unit takes the form of an electronic and computer system comprising, for example, a microprocessor designed to execute a command program. Execution of this program enables the control unit to control the light source(s), depending on the signals received by the optical sensor(s). In particular, the control unit determines the dose delivered by integrating, at regular time intervals, the light intensity detected by the optical sensors during said time interval, up to the target irradiation dose.

The apparatus also includes an information collection device 34. This information collection device 34 is configured to collect information regarding a parameter of the irradiation receptacle 2.

According to an embodiment, the information collection device 34 is a man-machine interface enabling the operator to manually enter an inscription comprising the information regarding the transmittance of the irradiation receptacle 2.

Alternatively, the information collection device 34 is an information reader, such as a camera, a barcode reader or a RFID reader.

For example, this information reader 34 is fixedly arranged in the recess 22 of the irradiation apparatus 21 so that it can read the information from the irradiation receptacle 2 when the latter is arranged in said recess 22.

In one embodiment, the information reader 34 is arranged opposite the information storage and retrieval means of the irradiation receptacle 2.

For example, the information reader 34 is a mobile hand-held information reader, such as a mobile hand-held barcode reader. In this case, the operator reads the information from the irradiation receptacle 2 prior to irradiation treatment.

Alternatively, the information reader is a fixed barcode reader positioned opposite the barcode integrating the information regarding a parameter of the irradiation receptacle.

In another particular embodiment, the information collection device is a device for performing a spectrophotometric measurement on a wall of the irradiation receptacle to determine its transmittance.

According to the invention and in order to ensure that the biological fluid is subjected to the predetermined irradiation dose, the control unit of the apparatus is configured to (i) obtain information regarding a parameter of the irradiation receptacle, (ii) determine a target irradiation dose on the basis of said information regarding said parameter and the predetermined irradiation dose, and (iii) irradiate the irradiation receptacle with said target irradiation dose.

According to the invention, information regarding a parameter of the irradiation receptacle comprises information including the transmittance value of a wall of the irradiation receptacle. In particular, the information comprises the value of the UV or UV-C transmittance of a wall of the receptacle. Even more particularly, this information is the value of the UV or UV-C transmittance of a wall of the receptacle, as described above.

The control unit of the apparatus is configured to obtain this information regarding a parameter of said irradiation receptacle. In particular, this information is collected by the information collecting device 34 and transmitted to the control unit for processing.

The control unit then determines a target irradiation dose, taking into account the information about the transmittance value of the wall of the receptacle and the predetermined irradiation dose. The target irradiation dose is a dose calculated and corrected in particular as a function of the UV and/or UV-C transmittance of a wall of the irradiation receptacle.

For example, if the transmittance of the wall of the irradiation receptacle is lower than that with which the predetermined dose was determined, the target irradiation dose delivered by the apparatus will be higher.

In a particular embodiment, further information regarding the irradiation receptacle and/or the biological fluid to be irradiated is taken into account to determine the target irradiation dose.

In particular, the applicant has found in numerous tests that certain manufacturing steps, such as packaging of the finished product and sterilization, can have a negative impact on the UV and/or UV-C transmittance of the irradiation receptacle.

For example, when the information regarding a parameter of the irradiation receptacle is the transmittance value of the sheath or sheets forming the irradiation receptacle before manufacture and/or packaging and/or sterilization of the receptacle, a correction coefficient is applied to this initial transmittance value in order to take into account the impact of the manufacturing steps of the receptacle.

This correction coefficient is predetermined. Alternatively, this correction coefficient is variable information relating to the transmittance of the wall of the irradiation receptacle, which is integrated into the information storage and retrieval means described above.

Information regarding the biological fluid that can influence irradiation includes for example the volume, the platelet concentration or the plasma quantity.

Once the target irradiation dose has been calculated, the control unit is programmed to irradiate the irradiation receptacle with said target irradiation dose using the light source(s) of the apparatus.

In this way, it is more certain that the biological fluid receives the necessary and sufficient dose of irradiation to inactivate pathogens, without adversely affecting the biological properties of the biological fluid.

In practice, the method for operating the apparatus 21 comprises the following steps:
a) placing the irradiation receptacle 2 containing the biological fluid to be treated in the recess 22 of the apparatus 21,
b) obtaining, with the aid of the information collection device 34, said information, that comprises information comprising the value of the transmittance of a wall of the irradiation receptacle 2,
c) determine a target irradiation dose from said information regarding said parameter and said predetermined dose, and
d) irradiate the irradiation receptacle 2 so as to irradiate the biological fluid with said target irradiation dose, with the light source(s) 23,24 of the apparatus 21.

Another aspect of the invention is a system comprising on the one hand an apparatus for the treatment by electromagnetic irradiation at a predetermined dose of a biological fluid contained in an irradiation receptacle and on the other hand an irradiation receptacle as described above.

In one embodiment, the information collection device 34 of the apparatus 21 is an information reader, such as a camera, a barcode reader or a RFID reader. This information reader is fixedly arranged in the recess 22 of the irradiation apparatus 21, so that it can read the information from the irradiation receptacle 2 when the latter is arranged in said recess 22.

In the same mode, the information regarding the transmittance of a wall of the irradiation receptacle 2 is included in an information storage and retrieval means such as an inscription, a bar code, a QR code or an RFID chip.

For example, the information reader is arranged opposite the information storage and retrieval means.

For example, the information reader is a fixed barcode reader positioned opposite a barcode integrating information regarding a parameter of the irradiation receptacle.

1. A method for the treatment by electromagnetic irradiation at a predetermined dose of a biological fluid contained in an irradiation receptacle (2), said irradiation receptacle having at least one wall permeable to said electromagnetic radiation, the method comprising the following steps:
(i) obtaining information regarding a parameter of said irradiation receptacle,
(ii) determining a target irradiation dose on the basis of said information regarding said parameter and said predetermined dose, and
(iii) (iii) irradiating the irradiation receptacle with said target irradiation dose, said method being characterized in that said information regarding the parameter of the irradiation receptacle comprises the transmittance value of the wall of the irradiation receptacle.

2. Method according to claim 1, characterized in that the information regarding a parameter of said receptacle is included in an information storage and retrieval means.

3. Method according to claim 2, characterized in that the information storage and retrieval means is a bar code (7).

4. Method according to claim 1, characterized in that the electromagnetic radiation is UV or UV-C radiation.

5. Method according to claim 1, characterized in that the irradiation receptacle (2) is an irradiation bag comprising at least one wall of a flexible film made of a material permeable to electromagnetic radiation.

6. Method according to claim 1, characterized in that the biological fluid is blood or a blood component.

7. Apparatus (21) for carrying out the method according to claim 1, said apparatus comprising a recess (22) configured to receive said irradiation receptacle (2) containing the biological fluid, one or more light sources (23, 24) emitting said electromagnetic radiation, an information collection device (34) configured to collect information regarding a parameter of said irradiation receptacle, and a control unit configured to (i) obtain information regarding a parameter of said irradiation receptacle, (ii) determine a target irradiation dose on the basis of said information regarding said parameter and said predetermined dose, and (iii) irradiate the irradiation receptacle with said target irradiation dose, characterized in that the information regarding the parameter of the irradiation receptacle comprises the transmittance value of the wall of the irradiation receptacle.

8. Apparatus according to claim 7, characterized in that the information collection device (34) is an information reader.

9. Apparatus according to claim 7, characterized in that the information collection device (34) is a barcode reader.

10. Apparatus according to claim 7, characterized in that the recess (22) for receiving the irradiation receptacle (2) comprises a plate (25) on which the irradiation receptacle (2) is intended to be placed.

11. Apparatus according to claim 7, characterized in that it comprises two light sources (23, 24) on either side of said recess (22).

12. Irradiation receptacle (2) for use in the method according to claim 1, said irradiation receptacle having at least one wall permeable to electromagnetic radiation and comprising information regarding a parameter of said irradiation receptacle, characterized in that said information comprises the transmittance value of the wall of the irradiation receptacle.

13. System comprising an apparatus (21) according to claim 7, and an irradiation receptacle (2).
